(19) United States
(12) Patent Application Publication
(10) Pub. No.: US 2023/0112617 A1
(43) Pub. Date: Apr. 13, 2023

(54) ABSORBENT GARMENT

(71) Applicant: Hello Hazel, Inc., New York, NY (US)

(72) Inventors: Steven Cruz, New York, NY (US); Paul Davies, Wellingborough (GB); Aubrey Hubbell, New York, NY (US)

(21) Appl. No.: 17/965,605

(22) Filed: Oct. 13, 2022

Related U.S. Application Data
(60) Provisional application No. 63/255,431, filed on Oct. 13, 2021.

(51) Int. Cl. A61F 13/537; A61F 13/49

(52) U.S. Cl. CPC A61F 13/53747; A61F 13/53756; A61F 13/49011; A61F 13/49014; A61F 2013/53778; A61F 2013/530489

(57) ABSTRACT

An absorbent core structure comprising: a first piece of absorbent core material, a second piece of absorbent core material, an upper wicking layer, a lower wicking layer, and a barrier layer. The upper wicking material is located above the first and second piece of absorbent core material. The lower wicking layer is located below the first and second piece of absorbent core material. The absorbent core structure further includes a channel between the first and second piece of absorbent core material. The upper wicking layer is bonded to the lower wicking layer at a bottom portion of the channel and the barrier layer is bonded to the lower wicking layer.

### REFERENCE TO RELATED APPLICATIONS

This application claims the priority benefit of U.S. Provisional Patent Application No. 63/255,431, filed Oct. 13, 2021, the contents of which are incorporated herein by reference in their entirety.

### TECHNICAL FIELD

The present disclosure relates to absorbent garments and methods of making the same. Specifically, the present disclosure relates to single-use underwear made with super absorbent fibers.

### BACKGROUND

The human body is capable of discharging various fluids such as blood, urine, sweat, semen, and milk. Absorbent products for particular purposes, for instance, menstrual fluid or urine, have come a long way over the years in terms of the comfort and protection they offer the wearer. However, these products still look, feel, and wear like “articles” and not “clothing.” There is a need to make these articles more comfortable and less noticeable while providing a similar or better absorbent function.

In particular, incontinence is a condition which effects a large percentage of women during their lives. There are several causes, ranging from childbirth to obesity, which can lead to incontinence, and, for many women, there is no effective treatment to reverse the condition and therefore women must manage by wearing absorbent products (pads or adult diapers) to absorb urine leaks. These solutions, whilst effective, can be big and bulky and force the wearer to change the clothes they wear to hide or disguise the absorbent product. As incontinence has historically been seen as an embarrassing condition that people don't discuss, being forced to change clothes and behaviors has contributed to women feeling depressed and isolated after becoming incontinent.

As absorbent products are large and bulky, they can be visible when worn under tight fitting clothes such as jogging pants. Recent developments with advanced fabrics in, for example, sports clothing, has allowed designers to create reusable underwear that can be worn during menstruation and provide protection without needing an additional absorbent product to be worn in the underwear. These products deliver on the desired thinness and discretion, but they lack the necessary level of absorbency to protect women with incontinence.

There is a need for an underwear garment that retains the thinness and discretion of regular underwear whilst offering comparable levels of protection to absorbent pads and adult diapers. In addition to thinness and discretion, the garment needs to be cut to fit the body like regular underwear and look like regular underwear, rather than looking like a larger diaper.

There is a need for a garment capable of absorbing around 1 cup of urine. SAP (super absorbing polymer) is commonly used to absorb and lock away urine in currently available products. However, because SAP requires significant energy input to remove liquid and return it to its dry condition, using SAP in a reusable garment would render it practically impossible to wash at home and therefore, with currently available technology, garments containing super absorber are suitable only as a single use disposal products. To create a single use absorbent underwear garment, there are several additional technical challenges posed by the choice of materials.

Accordingly, a need exists for an absorbent garment which is both comfortable and functional. The present disclosure focuses on using a core material that is different than an SAP which allows for the creation of different core designs as the material can be cut into different shapes. Additionally, channels, holes and other patterns can be cut into the core material of the present disclosure and finally the core material of the present disclosure can be stacked to create different absorption zones within the core.

### SUMMARY

The present disclosure is directed to an absorbent core structure that overcomes one or more of the aforementioned shortcomings of the prior art. The absorbent core structure comprises super absorbent fibers

One embodiment of the present disclosure teaches an absorbent core structure comprising: a first piece of absorbent core material; a second piece of absorbent core material; an upper wicking layer located above the first and second piece of absorbent core material; a lower wicking layer located below the first and second piece of absorbent core material; and a barrier layer wherein there is a channel between the first and second piece of absorbent core material, wherein the upper wicking layer is bonded to the lower wicking layer at a bottom portion of the channel; and wherein the barrier layer is bonded to the lower wicking layer.

Another embodiment of the present disclosure teaches an absorbent garment comprising: an elasticated outer garment layer; an absorbent core structure comprising: a first piece of absorbent core material; a second piece of absorbent core material; an upper wicking layer located above the first and second piece of absorbent core material; a lower wicking layer located below the first and second piece of absorbent core material; and a barrier layer wherein there is a channel between the first and second piece of absorbent core material, wherein the upper wicking layer is bonded to the lower wicking layer at a bottom portion of the channel; and wherein the barrier layer is bonded to the lower wicking layer; a cover retaining the absorbent core structure within the absorbent garment; wherein the cover is ultrasonically welded to the elasticated outer garment layer.

### BRIEF DESCRIPTION OF THE DRAWINGS

The drawings and detailed description that follow are intended to be merely illustrative and are not intended to limit the scope of the present disclosure as contemplated by the inventors. The detailed description of specific embodiments of the present disclosure can be best understood when read in conjunction with the following drawings.

FIG. 1 is a side view showing the super absorbing fibers (“SAF”) material flexibility;

FIG. 2 is a top-perspective view illustrating how different layers of core material can be stacked to create zones of higher absorbency;

FIG. 3 is a cross-section view showing two separate pieces of core material with a channel separating the two separate pieces of core material;

FIG. 4 is a side view showing an example of how a single piece of core material expands in the z-direction after absorption of fluid.

FIG. 5 is a cross-section view showing the two separate pieces of core material as shown in FIG. 3 when said two separate pieces of core material absorb fluid;

FIG. 6 is a cross-section view showing an absorbent core structure with a wicking later bonded in the channel between the two pieces of core material as shown in FIG. 3;

FIG. 7A is a cross-section view showing the absorbent core structure of FIG. 6 as it absorbs a fluid;

FIG. 7B is a cross-section view showing the absorbent core structure of FIG. 7A after it absorbs a fluid;

FIG. 8 is a cross-section view showing an absorbent core structure of the present disclosure that includes an upper wicking layer within a channel between two separate pieces of core material, a lower wicking layer located below the two separate pieces of core material; and a barrier layer located below the lower wicking layer;

FIG. 9 is a cross-section view showing how the barrier layer of the absorbent core structure of FIG. 8 being wrapped up and around the structure, but as not to overlap the upper wicking layer;

FIG. 10 is a cross-section view showing how the various elements of the absorbent core structure are secured to one another;

FIG. 11 is a top view of the absorbent core structure showing the barrier layer secured over the top portion of said absorbent core structure;

FIG. 12 is a cross-section view showing an embodiment of an absorbent garment;

FIG. 13 is a front perspective view of an embodiment of an absorbent garment; and

FIG. 14 depicts a step-by step folding process utilized to fold an absorbent garment.

### DETAILED DESCRIPTION

In its broadest aspects, embodiments of the present disclosure are directed to an absorbent garment that overcomes one or more of the aforementioned shortcomings of the prior art. While the disclosed embodiments of the present disclosure satisfy long-felt but unmet needs, it should be understood that the garment is not limited to being implemented in the precise manners set forth herein, but could be implemented in other manners without undue experimentation by those of ordinary skill in the art in light of this disclosure. Accordingly, the examples set forth herein should be understood as being illustrative only, and should not be treated as limiting.

Firstly, textiles can stretch in both the X and Y plane to ensure a good fit against the wearers body, but textiles are too expensive to be used in a single use product. Nonwovens are cheaper to produce and therefore more suited for use in single use products, but they typically only stretch in one plane. Therefore, to ensure a good fit to the wearer's body, additional elastics need to be added to the garment, especially around the leg openings. The addition of elastic tapes to create leg and waist elasticated seams is standard in the manufacture of underwear and only required that an appropriate elastic tape is selected to deliver the required degree of stretch.

Secondly, it is also necessary to choose nonwovens for the different parts of the garment with similar stretch characteristics, to ensure all parts of the garment move and flex to the same degree, as the wearer's body moves. This was resolved by using the same material for the garment and for the cover.

Finally, the materials chosen for the absorbent core within the garment do not stretch by design, and therefore the core has to be incorporated into the garment in such a way to ensure that the core is securely bonded into the garment and that the core does not prevent the garment from stretching and flexing as this could be both uncomfortable for the wearer and make the garment visible when worn with tight-fitting outer clothes.

To maintain the desired discretion of the garment, the absorbent structure needs to be as thin as possible and the same width as typical fabric underwear. Many commercially available absorbent pads and adult diapers use a mixture of super absorbing polymer (“SAP”) and fluff pulp fibers to create a highly effective absorbent structure. However, a mixture of SAP and fluff pulp fibers is not sufficiently thin or flexible and is therefore not sufficiently narrow enough for the product construction in mind. Additionally, SAP and fluff pulp fibers are typically manufactured in-line with the production of the pad or adult diaper, and this necessitates purchase of complex and expensive manufacturing equipment.

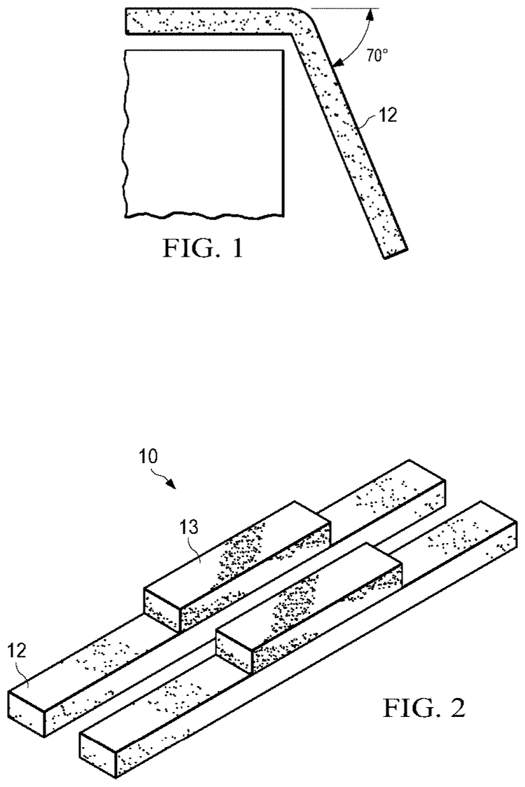

To meet the design requirements of the absorbent garment 100 of the present disclosure, super absorbing fibers (“SAF”) were selected as opposed to SAPs. SAF can be formed into thin and flexible nonwovens which can be manufactured offline and supplied on a roll, which provides SAF inherent advantage for use in the absorbent garments 100 of the present disclosure. For the absorbent garments 100 of the present disclosure, to ensure the absorbent core structure 10 is as thin, flexible, and conformable as possible, a needle-punch nonwoven containing SAF fibers is utilized for the absorbent core 12. It is possible to create an absorbent core 12 that provides high levels of liquid absorbency. Needle-punched materials can be made to have good strength and flexibility as the process used to entangle the fibers still allows them to move relative to each other. As shown in FIG. 1, the absorbent core 12 utilized in the absorbent core structure 10 of the present disclosure have a flexibility of at least 70 degrees when measured against a horizontal plane.

As the absorbent core is a piece of nonwoven, it offers the advantage that it can be easily cut and placed within a garment, but it also allows for different shapes to be used and for gaps or spaces to be introduced into the absorbent core structure 10 to aid fluid movement. For example, the absorbent core 12 can be discontinuous or can have one of more channels or holes cut in the structure, along the length of the core or side to side. In addition, as shown in FIG. 2, it is also possible to stack additional pieces of absorbent core material 13 in the absorbent core structure 10 to create zones or areas of higher protection within an absorbent garment. These additional pieces of core material can cover the whole length of the absorbent core structure 10, or only part of it, for example, in the center of a crotch area of an absorbent garment where liquid will initially hit the absorbent core structure 10. Additionally, these additional pieces of absorbent core material 13 can be of the same composition as the main core 12 or a different composition. The core material may form different core designs easily as the material can be cut into different shapes. Additionally, channels, holes and other patterns can be cut into it and finally it can be stacked to create different absorption zones within the core.

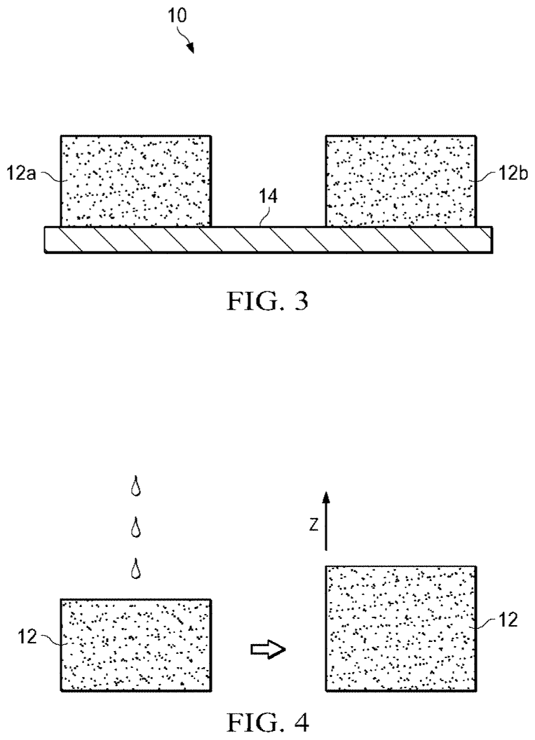

In one or more embodiments, the absorbent core structure 10 utilizes a rectangular absorbent core 12. In one or more embodiments, such as shown in FIG. 3, the rectangular absorbent core is separated into two pieces 12a and 12b and are placed into the absorbent core structure with a gap/channel 14 in between the two pieces 12a and 12b. In one or more embodiments, the gap/channel 14 is between 2 mm and 10 mm in width. In one embodiment, the width of the gap/channel 14 is 5 mm. the first piece of absorbent core material 12a has an outer edge 11a and an inner edge 11b and wherein the second piece of absorbent core material 12b also has an outer edge 11a and an inner edge 11b. The inner edge 11b of the first piece of absorbent core material 12a being adjacent the inner edge 11b of the second piece of absorbent core material 12b. In one or more embodiments, the width between the outer edge 11a of the first absorbent core material 12a and the outer edge 11a of the second absorbent core material 12b is greater than 60 mm, in other embodiments greater than 65 mm, in other embodiments greater than 70 mm, and in yet other embodiments greater than 75 mm. In one or more embodiments, the width between the outer edge 11a of the first absorbent core material 12a and the outer edge 11a of the second absorbent core material 12b is between 60 mm and 80 mm, in other embodiments between 65 mm and 80 mm, in other embodiments between 70 mm and 80 mm, and in yet other embodiments between 75 mm and 80 mm.

The gap/channel 14 aids fluid movement along the length of the absorbent core structure 10. However, initial experiments revealed two problems with this construction. Firstly, because the material of the absorbent core 12 is made of individual fibers, when it absorbs fluid, the fibers expand and change their shape and orientation. The expansion is predominately in the z-direction (out of the plane of the core) and thus has the effect of causing the core to shrink across its width, as shown in FIG. 4. This behavior is undesirable as a reduction in the width of the absorbent core 12 will result in local saturation which can lead to leakage. To improve the performance of the absorbent core 12, non-absorbent fibers were added in the range 50-10% of the total absorbent core material, thus reducing the observed shrinkage of the absorbent core 12. Further experimentation determined that optimum fluid performance was obtained utilizing between 20-30% non-absorbing fibers within the absorbent core. In one or more embodiments, the non-absorbing fibers are selected from the group consisting of polyester fibers, polypropylene fibers, polylactic acetate fibers, and combinations thereof. Additionally, it was determined that only one grade of SAF fibers should be used within each layer of absorbent core 12, wherein the grade of the SAF fibers refers to the absorption rate of the SAF fibers. However, if multiple layers of absorbent core material 12 were utilized, the grade of the SAF fibers could vary from layer to layer.

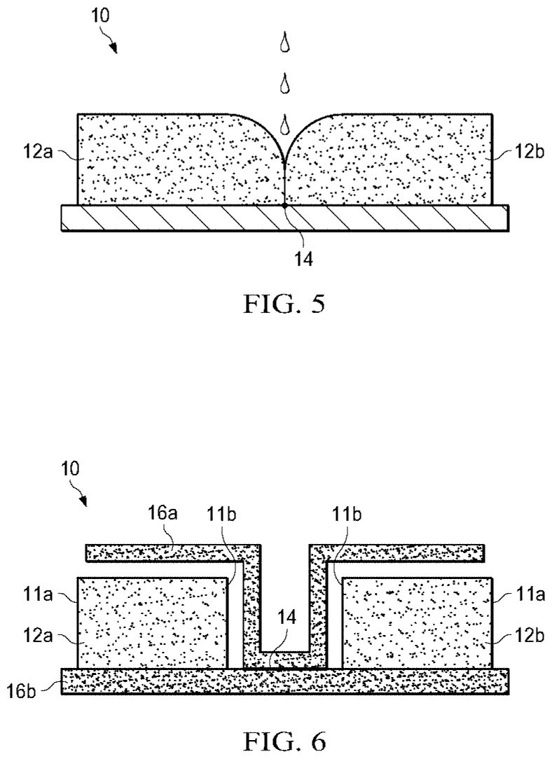

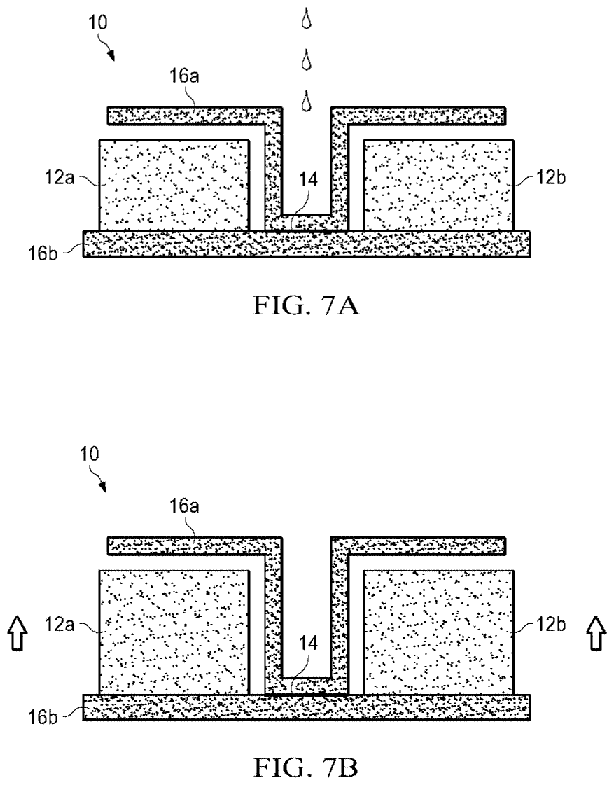

Secondly, it was observed that the SAF fibers tend to expand rapidly upon absorption of fluids. This rapid expansion lead to the formation of a gel-like material which tended to flow into any channels or gaps adjacent the absorbent core 12. In embodiments of the present invention wherein the rectangular absorbent core 12 is separated into two pieces 12a and 12b, this gel would flow into the channel 14 between the two pieces 12a and 12b, preventing any further fluid from entering the channel 14. This formation of a gel-like material in the channel 14 between the two pieces 12a and 12b is shown in FIG. 5. This problem was solved by bonding a nonwoven wicking layer to the top 16a and bottom 16b of the core material and then bonding these two separate layers together at the bottom portion of the channel as shown in FIG. 6. This effectively creates a pocket that the core material sits inside. This construction allows the core to expand and absorb liquid, but it stops any blockage of the channel, allowing liquid to be rapidly absorbed and transported along the length of the product as shown in FIGS. 7A and 7B. Different types of nonwovens containing either cellulosic or synthetic fibers are suitable to create the upper wicking layer 16a and the lower wicking layer 16b and different materials could be selected for the upper 16a and lower 16b layers within one absorbent core structure 10. In one or more embodiments of the present disclosure, an airlaid nonwoven containing at least 70% cellulosic fibers was selected for both the upper 16a and lower 16b layers. In one or more embodiments, the cellulosic fibers can be selected from the group consisting of fluff pulp fibers, rayon fibers, lyocell fibers or natural fibers such as cotton, and combinations thereof.

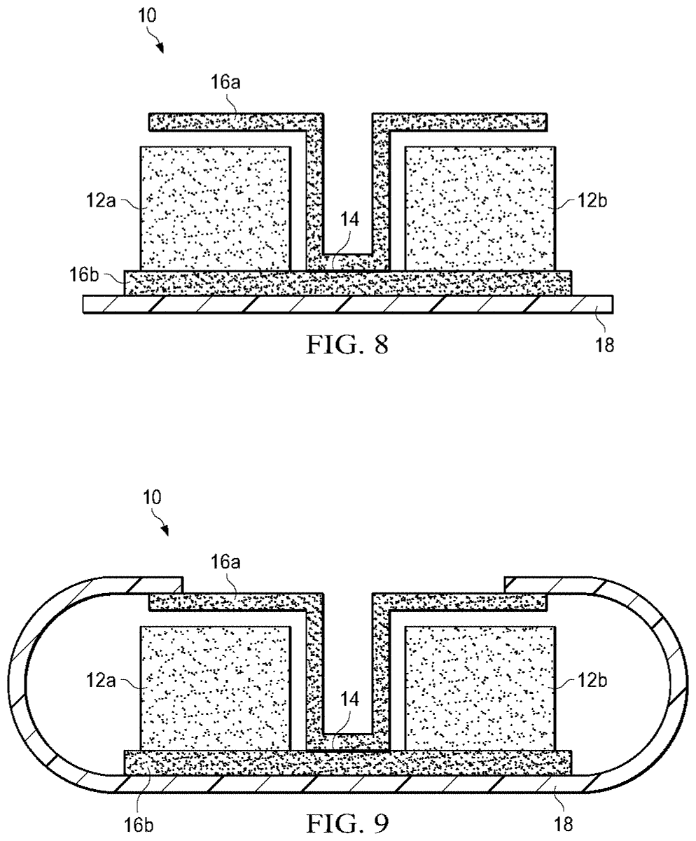

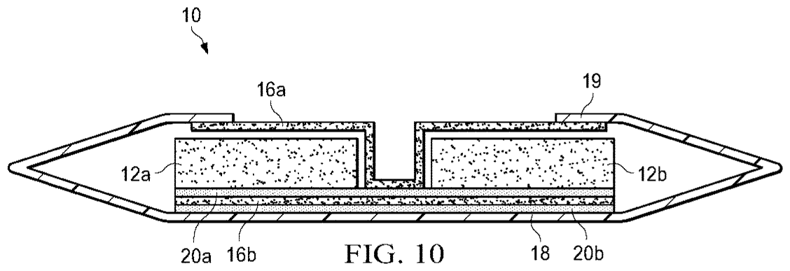

In one or more embodiments, as shown in FIG. 8, the absorbent core structure 10 contains a plastic barrier layer 18 located on the bottom most portion of the absorbent core structure. In one or more embodiments, the barrier layer 18 is wrapped around the absorbent core 12 and extends beyond the width of the absorbent core structure 10 to allow the barrier layer 18 to bond into leg seams of a garment (not shown) as shown in FIG. 9. This folding over of the barrier layer 18 creates a fin 19 on either side of the absorbent core structure. It was also determined that extending the barrier layer 18 into the side seams of the absorbent garment 100 was preferable to avoid creating gaps between the absorbent core structure 10 and leg seams of the absorbent garment 100 which could result in leakage. The barrier layer 18 can be included in the side seam for the entire length of the absorbent core structure 10, or only part of the length of the absorbent core structure 10. In one or more embodiments the barrier layer 18 is selected from the group consisting of polyethylene, polypropylene, polyester, polylactic acetate, and combinations thereof.

The elements of the absorbent core structure 10 will need a means to bond to one another. As shown in FIG. 10, there is a first layer of adhesive 20a between the upper 16a and lower 16b wicking layers to bond these two layers together within the channel 14 between the two pieces of absorbent core material 12a and 12b. This layer of adhesive 20a is also used to secure the two pieces of absorbent core material 12a and 12b to the lower wicking layer 16b. As shown in FIG. 10, a second layer of adhesive 20b bonds the lower wicking layer 16b to the barrier layer 18. It is important to note that there should be minimal adhesive found on the top surface of the absorbent core structure 10 so as not to impede and absorption of liquid by the absorbent core material 10.

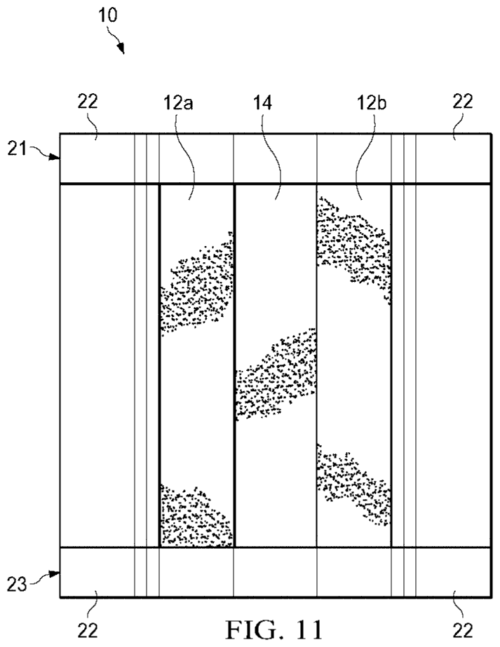

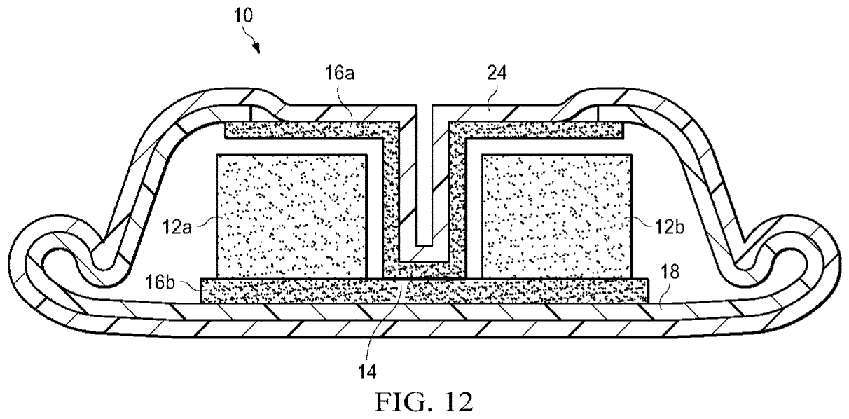

As shown in FIG. 11, it is only contemplated that a minimal amount of adhesive 22 is found at the ends 21 and 23 of the barrier layer 18 to secure the barrier layer 18 to the absorbent core structure 10. This will be the only portion of the absorbent core structure 10 that is against the wearer's skin wherein adhesive will be found.

The absorbent core structure 10 needs to be retained underneath a cover 24. The function of the cover 24 is twofold: (i) be soft against the wearer's skin and match the style of the garment 100 and (ii) allow liquid to pass quickly into the absorbent core structure 10 and mask any stains from the liquid. The types of nonwovens typically used as covers in absorbent garments are not flexible. However, it is an object of the absorbent garment 100 of the present disclosure to utilize a material for the cover 24 that is flexible and allows for good liquid transmission. In one or more embodiments, the material for the cover 24 is selected from the group consisting of elasticated polypropylene spunbonded material, elasticated spunbounded polyester material, elasticated air through bonded nonwoven material, elasticated Spunbond Meltblown Spunbond (SMS) nonwoven material, elasticated carded material, and elasticated thermally bonded nonwoven material, and combinations thereof. In one embodiment, the cover 24 is made from an elasticated polypropylene spunbonded material, which, when tested, met all the criteria for elasticity and fluid absorbency.

The initial idea was to bond the cover 24 to the sides of the garment 100 and allow it to float over the top of the absorbent core structure 10. Experiments with this construction revealed a problem. If the cover 24 is not in close contact with the upper wicking layer 16a of the absorbent core structure 10, liquid will tend to stay in the cover 24, leading to the cover 24 feeling wet against the wearer's skin. To solve this problem, it was necessary to fully bond the cover 24 to the absorbent core structure 10. However, this in turn created an additional problem. The absorbent core structure 10 was now bonded to both the outer layer of the garment 100 and to the cover 24.

As nonwoven materials only stretch across the garment 100 and not along the crotch area, as the garment 100 is formed, the inner and outer layers of the garment 100 were unable to move to the same degree, leading to the absorbent core structure 10 creasing and deforming and creating ridges in the garment 100. In addition, if the absorbent core structure 10 is too tightly bonded together, the core 12 would be unable to expand in the z-direction when fluid is absorbed. To solve the first problem, in some embodiments of the present disclosure, the absorbent core structure 10 is allowed to float against the outer layer of the garment 100 by not bonding the barrier layer 18 to the outer layer of the garment 100, and the barrier layer 18 is retained by the formation of the leg seams of the garment 100. However, in other embodiments, it is possible to bond the barrier layer 18 to the outer layer of the garment 100 in the center of the crotch only (as this part of the garment 100 experiences the least movement) or to bond the barrier layer 18 to the outer layer of the garment 100 discontinuously along where the barrier layer 18 meets the cover 24.

To solve the second problem, the upper nonwoven wicking layer 16a is not bonded to the upper surface of the absorbent core 12 and 12b, and the barrier layer 18 is also not bonded to the absorbent core 12a and 12b or the upper wicking layer 16a along the long axis of the absorbent core structure 10. This creates an unbonded zone between the cover 24 and the absorbent core structure 10, creating a zone where the absorbent core 12a and 12b can expand in the z-direction as a result of the unbonded zone and the elasticated cover 24. In one or more embodiments, the upper wicking layer 16a could be bonded to the top surface of the absorbent core 12a and 12b either continuously or discontinuously along the long axis of the absorbent core structure 10, if the construction ensured that the barrier layer 18 remained unbonded to the top surface of the absorbent core 12a and 12b, and if the barrier layer 18 does not overlap the upper wicking layer 16a.

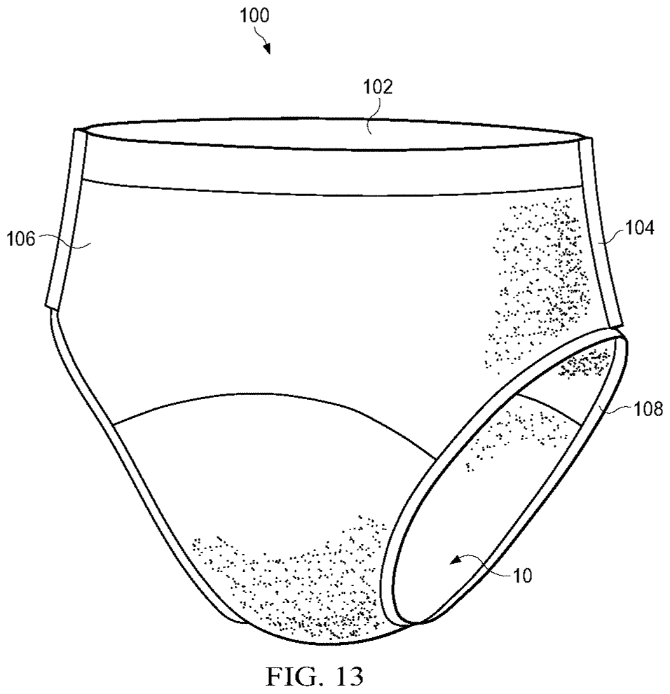

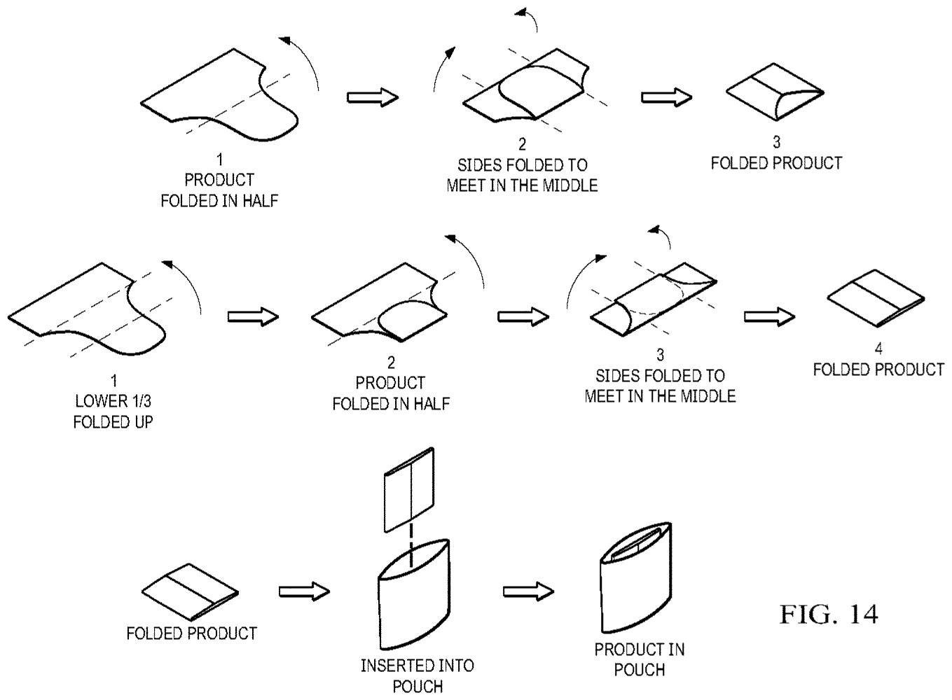

FIG. 13 shows one embodiment of the absorbent garment 100 of the present disclosure. The absorbent garment 100 also includes an elasticated waist band 102, side seams 104, elasticated outer layer 106, and elasticated leg seams 108. The elasticated outer layer 106 has a hydrophilic and a hydrophobic side, wherein the hydrophilic side should face inside the garment 100 and the hydrophobic side should form the outside of the garment 100. The elasticated waist band 102 and the elasticated leg seams 108 are bound to the elasticated outer layer 106 before the absorbent core structure 10 and cover 24 are positioned within the garment 100. Once the cover 24 is placed around the absorbent core structure 10 as discussed above, the cover 24 containing the absorbent core structure is ultrasonically welded to the elasticated outer layer 106. The absorbent garment 100 is folded utilizing any of the combination of steps shown in FIG. 14 to fit the absorbent garment 100 into a “purse pack” shape to enable it to always be handy. This method of folding the absorbent garment 100 allows for it to fit nicely into a small pouch. In one or more embodiments, the absorbent garment 100 is compressed before packaging. This can help with the sustainability equation. The absorbent garment 100 may be sold in various sizes and colors. In one or more embodiments, the absorbent garment 100 may comprise side seams 104 that are permanent or they may be rippable to enable easy removal of a soiled absorbent garment 100.

In one or more embodiments, the absorbent core structure 10 of the present disclosure has an absorbent capacity of greater than 7.5 g/g, in other embodiments greater than 10 g/g, in other embodiments greater than 15 g/g, in other embodiments greater than 20 g/g. In one or more embodiments, the absorbent core structure 10 of the present disclosure has an absorbent capacity of between 7.5 g/g and 2 5g/g, in other embodiments between 10 g/g and 25 g/g, in other embodiments between 15 g/g and 25 g/g, and in yet other embodiments between 15 g/g and 20 g/g.

In one or more embodiments, the absorbent core structure 10 of the present disclosure can absorb greater than 100 mL, in other embodiments greater than 150 mL, in other embodiments greater than 200 mL, and in yet other embodiments greater than 225 mL. In one or more embodiments, the absorbent core structure 10 of the present disclosure can absorb between 100 mL and 235 mL, in other embodiments between 150 and 235 mL, and in yet other embodiments between 200 and 235 mL. In one embodiment, the absorbent core structure 10 can absorb up to 235 mL.

In one or more embodiments, the absorbent core structure 10 of the present disclosure has an absorbency speed of less than 45 seconds, in other embodiment less than 35 seconds, in other embodiments less than 30 seconds, in other embodiments less than 25 second, and in yet other embodiment less than 20 seconds when absorbing 100 mL of liquid measured using test method NWSP070.9.R1(15). In one or more embodiments, the absorbent core structure 10 of the present disclosure has an absorbency speed of between 45 seconds and 15 seconds, in other embodiments between 35 seconds and 15 seconds, in other embodiments between 30 second and 15 seconds, in other embodiments between 25 seconds and 15 seconds, and in yet other embodiments between 20 seconds and 15 seconds when measured using test method NWSP070.9.R1(15). In one embodiment, the absorbent core structure 10 has an absorbency speed of 15 seconds when absorbing 100 mL of liquid measured using test method NWSP070.9.R1(15).

In one or more embodiments, the absorbent core structure 10 of the present disclosure has a core utilization when wet of greater than 45%, in other embodiments greater than 50%, in other embodiments greater than 55%, in other embodiments greater than 60%, and in yet other embodiments greater than 65%. In one or more embodiments, the absorbent core structure 10 of the present disclosure has a core utilization when wet of between 45% and 70%, in other embodiments between 50% and 70%, in other embodiments between 55% and 70%, in other embodiments between 60% and 70%, and in yet other embodiments between 65% and 70%. In one embodiment, the absorbent core structure 10 has a core utilization when wet of 70%.

In one or more embodiments, the absorbent core structure of the present disclosure has a thickness when dry of less than 15 mm, in other embodiments less than 12.5 mm, in other embodiments less than 10 mm, and in yet other embodiments less than 7.5 mm. In one or more embodiments, the absorbent core structure of the present disclosure has a thickness when dry of between 15 mm and 6 mm, in other embodiments between 12.5 mm and 6 mm, in other embodiments between 10 mm and 6 mm, and in yet other embodiments between 7.5 mm and 6 mm. In one embodiment, the absorbent core structure has a thickness when dry of 6 mm.

In one or more embodiments, the absorbent core 12 of the absorbent core structure 10 the present disclosure has a drapability from a horizontal plane of greater than 50 degrees, in other embodiments greater than 60 degrees, in other embodiments greater than 70 degrees, and in yet other embodiments greater than 80 degrees. In one or more embodiments, the absorbent core 12 of the absorbent core structure 10 of the present disclosure has a drapability from a horizontal plane of between 50 degrees and 85 degrees, in other embodiments between 60 degrees and 85 degrees, in other embodiments between 70 degrees and 86 degrees, and in yet other embodiments between 80 degrees and 85 degrees.

In one or more embodiments, the absorbent core structure 10 of the present disclosure has a total width of less than 14 cm, in other embodiments less than 12 cm, in other embodiments less than 10 cm, and in yet other embodiments less than 8 cm. In one or more embodiments, the absorbent core structure 10 of the present disclosure has a width of between 14 cm and 6 cm, in other embodiments between 12 cm and 6 cm, in other embodiments between 10 cm and 6 cm, and in yet other embodiments between 8 cm and 6 cm.

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

In various embodiments disclosed herein, a single component can be replaced by multiple components and multiple components can be replaced by a single component to perform a given function or functions. Except where such substitution would not be operative, such substitution is within the intended scope of the embodiments.

The foregoing description of embodiments and examples has been presented for purposes of illustration and description. It is not intended to be exhaustive or limiting to the forms described. Numerous modifications are possible in light of the above teachings. Some of those modifications have been discussed, and others will be understood by those skilled in the art. The embodiments were chosen and described in order to best illustrate principles of various embodiments as are suited to particular uses contemplated. The scope is, of course, not limited to the examples set forth herein, but can be employed in any number of applications and equivalent devices by those of ordinary skill in the art. Rather it is hereby intended the scope of the invention to be defined by the claims appended hereto. As such, the claims below shall be read to include all obvious variations and modifications that may be within the spirit of this invention.

What is claimed is:

1. An absorbent core structure comprising:
a. a first piece of absorbent core material;
b. a second piece of absorbent core material;
c. an upper wicking layer located above the first and second piece of absorbent core material;
d. a lower wicking layer located below the first and second piece of absorbent core material; and
e. a barrier layer
wherein there is a channel between the first and second piece of absorbent core material, wherein the upper wicking layer is bonded to the lower wicking layer at a bottom portion of the channel; and wherein the barrier layer is bonded to the lower wicking layer.

2. The absorbent core structure of claim 1, wherein the first and second piece of absorbent core material are made from a mix of super absorbent fibers and non-absorbent fibers and wherein the mix of super absorbent fibers and non-absorbent fibers includes from 10% to 50% of non-absorbent fibers.

3. The absorbent core structure of claim 2, wherein the non-absorbing fibers are selected from the group consisting of polyester fibers, polypropylene fibers, polylactic acetate fibers, and combinations thereof.

4. The absorbent core structure of claim 1, wherein the absorbent core structure has an absorbency speed of between 45 seconds and 15 seconds when absorbing 100 mL of liquid measured using test method NWSP070.9.R1(15).

5. The absorbent core structure of claim 1, wherein the upper and lower wicking layer is an airlaid nonwoven containing at least 70% cellulosic fibers and wherein the barrier layer is made from a material selected from the group consisting of polyethylene, polypropylene, polyester, polylactic acetate, and combinations thereof.

6. The absorbent core structure of claim 1, wherein the channel is between 2 mm and 10 mm in width.

7. The absorbent core structure of claim 1, wherein the first and second piece of absorbent core material are bonded to the lower wicking layer.

8. The absorbent core structure of claim 1, wherein ends of the barrier layer fold over a top portion of the first and second piece of absorbent core material to form a fin on each side of the absorbent core structure.

9. The absorbent core structure of claim 1, wherein the first piece of absorbent core material has an outer edge, wherein the second piece of absorbent core material has an outer edge, and wherein the width between the outer edge of the first piece of absorbent core material and the outer edge of the second piece of absorbent core material is between 70 mm and 80 mm.

10. An absorbent garment comprising:
a. an elasticated outer garment layer;
b. an absorbent core structure comprising: and
i. a first piece of absorbent core material;
ii. a second piece of absorbent core material;
iii. an upper wicking layer located above the first and second piece of absorbent core material;
iv. a lower wicking layer located below the first and second piece of absorbent core material; and
v. a barrier layer
c. a cover retaining the absorbent core structure within the absorbent garment;
wherein there is a channel between the first and second piece of absorbent core material, wherein the upper wicking layer is bonded to the lower wicking layer at a bottom portion of the channel, wherein the barrier layer is bonded to the lower wicking layer, and wherein the cover is ultrasonically welded to the elasticated outer garment layer.

11. The absorbent garment of claim 10, further comprising an elasticated waist band and elasticated leg seams.

12. The absorbent garment of claim 11, wherein the elasticated outer garment layer is folded in half and is ultrasonically welded together to form the absorbent garment.

13. The absorbent garment of claim 10, wherein the first and second piece of absorbent core material are made from a mix of super absorbent fibers and non-absorbent fibers and wherein the mix of super absorbent fibers and non-absorbent fibers includes from 10% to 50% of non-absorbent fibers.

14. The absorbent garment of claim 13, wherein the non-absorbing fibers are selected from the group consisting of polyester fibers, polylactic acetate fibers, and combinations thereof.

15. The absorbent garment of claim 10, wherein the upper and lower wicking layer is an airlaid nonwoven containing at least 70% cellulosic fibers and wherein the barrier layer is made from a material selected from the group consisting of polyethylene, polypropylene, polyester, polylactic acetate, and combinations thereof.

16. The absorbent garment of claim 10, wherein the channel is between 2 mm and 10 mm in width.

17. The absorbent garment of claim 10, wherein the first and second piece of absorbent core material are bonded to the lower wicking layer.

18. The absorbent garment of claim 10, wherein ends of the barrier layer fold over a top portion of the first and second piece of absorbent core material to form a fin on each side of the absorbent core structure.

19. The absorbent garment of claim 10, wherein the absorbent core structure has an absorbency speed of between 45 seconds and 15 seconds when absorbing 100 mL of liquid measured using test method NWSP070.9.R1(15).

20. The absorbent garment of claim 10, wherein the first piece of absorbent core material of the absorbent core structure has an outer edge, wherein the second piece of absorbent core material of the absorbent core structure has an outer edge, and wherein the width between the outer edge of the first piece of absorbent core material and the outer edge of the second piece of absorbent core material is between 70 mm and 80 mm.
